(19) United States
(12) Patent Application Publication
(10) Pub. No.: US 2012/0129149 A1
(43) Pub. Date: May 24, 2012

(54) REMOVAL OF OXYGEN FROM BIOLOGICAL FLUIDS

(76) Inventors: William J. Federspiel, Pittsburgh, PA (US); Tatsuro Yoshida, W. Newton, MA (US); Paul J. Vernucci, Billerica, MA (US); Brian Joseph Frankowski, Imperial, PA (US); Laura Wheeler Lund, Pittsburgh, PA (US)

(21) Appl. No.: 13/387,528

(22) PCT Filed: Jul. 31, 2010
(86) PCT No.: PCT/US10/44045

§ 371 Date: Jan. 27, 2012

Related U.S. Application Data
(60) Provisional application No. 61/230,575, filed on Jul. 31, 2009.

(51) Int. Cl. A61M 1/36

(52) U.S. Cl. 435/2; 422/44

(57) ABSTRACT

A system for reducing the concentration of oxygen in a fluid including red blood cells includes a housing, a plurality of hollow tubes extending within the housing and adapted for flow of the fluid therethrough, wherein each tube includes an inlet and an outlet, and a carrier system that reduces the concentration of oxygen at an exterior surface of the tubes to facilitate transport of oxygen from the fluid flowing through the tubes to an exterior of the tubes.

### CROSS-REFERENCE TO RELATED APPLICATIONS

This application claims benefit of U.S. Provisional Patent Application Ser. No. 61/230,575, filed Jul. 31, 2009, the disclosure of which is incorporated herein by reference.

### GOVERNMENTAL INTEREST

This invention was made with government support under grant no. R44HL088848-01 awarded by the National Institutes of Health Heart Lung and Blood Institute to New Health Sciences, Inc. The government has certain rights in this invention.

### BACKGROUND

The following information is provided to assist the reader to understand the technology described below and certain environments in which such technology can be used. The terms used herein are not intended to be limited to any particular narrow interpretation unless clearly stated otherwise in this document. References set forth herein may facilitate understanding of the technology or the background thereof. The disclosure of all references cited herein are incorporated by reference.

Stored blood and blood components (for example, fluid including or containing red blood cells), even if transfused within the current 6-week storage limit, deteriorate in a variety of significant ways including hemolysis (red cell destruction), low survival rate of transfused red cells in recipients, reduced deformability (inability to reach capillary beds), inability to release oxygen at tissue, and inability to dilate arterioles to increase perfusion.

Removal of oxygen from, for example, human red blood cell suspension (for example, blood collected in an anticoagulant solution and processed to remove platelets, white blood cells and other blood constituents) immediately after processing has been shown to extend the shelf life by 30%-100%. In addition to the extension of shelf life, a number of studies have shown that red blood cells stored under anaerobic conditions have higher ATP levels, lower hemolysis, and higher post-transfusion recovery compared to conventionally stored cells.

This improved efficacy, while beneficial in all circumstances, can, for example, particularly benefit subjects who require chronic transfusion therapy (for example, sickle cell disease or beta-thalassemia) by reducing transfusion frequency, time-averaged blood transfusion volume, and total iron burden. Moreover, an extended shelf-life improves the logistics of general blood banking, assists in alleviating periodic blood shortages, and enhances the utility of pre-operative autologous blood collection.

### SUMMARY

In one aspect, a system for reducing the concentration of oxygen in a biological fluid such as a fluid including red blood cells (for example, a fluid including a red blood cell suspension), includes a housing, a plurality of hollow tubes extending within the housing and adapted for flow of the fluid therethrough, wherein each tube includes an inlet and an outlet, and a carrier system that reduces the concentration of oxygen at an exterior surface of the tubes to facilitate transport of oxygen from the fluid flowing through the tubes to an exterior of the tubes.

The carrier system can, for example, include a fluid inlet in fluid connection with the housing, a fluid outlet in fluid connection with the housing and a system to circulate a fluid through a volume of the housing exterior to the tubes. The fluid that circulates through the volume of the housing can, for example, include a gas other than oxygen. The fluid inlet in fluid connection with the housing can, for example, be in fluid communication with a pump or with a source of the gas other than oxygen that is pressurized, or the fluid outlet in fluid connection with the housing can, for example, be in fluid communication with a vacuum source to circulate the gas other than oxygen through the volume of the housing.

The plurality of hollow tubes can, for example, be microporous tubes having tube diameters ranging from 150 microns to 200 microns. The plurality of hollow tubes can, for example, have pore diameters in the range of approximately 0.01 to 0.5 microns or in the range of approximately 0.1 to 0.4 microns.

The plurality of tubes can, for example, range in number from 5000 to 8000 tubes. The plurality of tubes can, for example, range in length from 10 cm to 50 cm.

In a number of embodiments, the carrier system includes an oxygen absorbing material. The oxygen absorbing material can, for example, be immobilized on an exterior surface of at least a portion of the tubes. The oxygen absorbing material can, for example, be positioned within a volume of the housing exterior to the tubes.

A number of the tubes can, for example, be arranged in a group and the absorbing material can surround at least a portion of a length of the group. A number of the tubes can be arranged in a plurality of groups and the absorbing material can surround at least a portion of a length of each of the plurality of groups. In a number of embodiments, a number of the tubes are arranged around a perimeter of the absorbing material over at least a portion of a length of the tubes.

The system can, for example, be connectible within a fluid path of a fluid processing system.

In another aspect, a method for reducing the concentration of oxygen in a biological fluid such as a fluid including red blood cell (for example, a fluid including a red blood cell suspension), includes flowing the fluid through a plurality of hollow microporous tubes extending within a housing, wherein a carrier system is in operative connection with the tubes to reduce the concentration of oxygen at an exterior surface of the tubes to facilitate transport of oxygen from the fluid flowing through the tubes to an exterior of the tubes.

In still a further aspect, a system for reducing the concentration of oxygen in a biological fluid such as fluid including red blood cells (for example, a fluid including a red blood cell suspension), includes a housing, a plurality of hollow tubes extending within the housing which include an oxygen absorbent material therein, an inlet in fluid connection with a volume of housing exterior to the tubes for entry of the fluid into the housing, and an outlet in fluid connection with a volume of housing exterior to the tubes for exit of the fluid from the housing.

The technology described herein, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a perspective, transparent view of an embodiment of an oxygen depletion device.

FIG. 1B illustrates a schematic representation of the oxygen depletion device of FIG. 1A.

FIG. 2A illustrates a perspective, partially transparent view of another embodiment of an oxygen depletion device.

FIG. 2B illustrates a transverse cutaway view of the oxygen depletion device of FIG. 2A.

FIG. 3A illustrates a perspective, partially transparent view of another embodiment of an oxygen depletion device.

FIG. 3B illustrates a transverse cutaway view of the oxygen depletion device of FIG. 3A.

FIG. 4A illustrates model predictions for average outlet pO₂ for various fiber length and numbers of fibers as well as measured average outlet pO₂ for a studied device as a function of device flow rate.

FIG. 4B illustrates a study of processing time as a function of device flow rate.

FIG. 4C illustrates code used to calculate the rate of oxygen transferred to the gas phase per unit volume.

FIG. 4D illustrates pO₂ as a function of flow rate for studies of several oxygen depletion devices.

FIG. 5A illustrates a side view of another embodiment of an oxygen depletion device.

FIG. 5B illustrates a top or end view of the device of FIG. 5A.

FIG. 5C illustrates a top or end view of a sorbent cartridge of the device of FIG. 5A.

FIG. 5D illustrates a side, cutaway view of a sorbent cartridge of the device of FIG. 5A.

FIG. 6A illustrates a side view of another embodiment of an oxygen depletion device.

FIG. 6B illustrates a transverse cutaway view of an embodiment of a hollow fiber and sorbent arrangement for use with the device of FIG. 6A.

FIG. 6C illustrates a transverse cutaway view of another embodiment of a hollow fiber and sorbent arrangement for use with the device of FIG. 6A.

FIG. 6D illustrates a transverse cutaway view of another embodiment of a hollow fiber and sorbent arrangement for use with the device of FIG. 6A.

FIG. 7 illustrates a schematic representation of the device of FIG. 1A wherein an inert carrier gas flows through the hollow fibers and red blood cell suspension flows through the volume exterior to the hollow fibers.

FIG. 8A illustrates a schematic representation of an embodiment of a oxygen depletion device in which red blood cell suspension flows around extending gas sorbent elements.

FIG. 8B illustrates an enlarged view of a gas sorbent element of FIG. 8A.

FIG. 9 illustrates schematically an embodiment of a blood processing (for example, including collection, processing and storage) system including an oxygen depletion device.

### DETAILED DESCRIPTION

As used herein and in the appended claims, the singular forms “a,” “an”, and “the” include plural references unless the content clearly dictates otherwise. Thus, for example, reference to “a sorbent” includes a plurality of such sorbents and equivalents thereof known to those skilled in the art, and so forth, and reference to “the sorbent” is a reference to one or more such sorbents and equivalents thereof known to those skilled in the art, and so forth.

In a number of embodiments, devices, systems and/or methods are described herein to remove a gas such as oxygen from biological fluids such as a fluid including red blood cells. In a number of representative embodiments described herein, oxygen is removed from red blood cell suspensions, red blood cell suspension products or red blood cell suspension components (human or otherwise). The term biological fluids as used in this application refers to fluid derived from biological sources (for example, from animals, including from humans). The term red blood cell suspensions as used in this application is defined as red blood cells suspended in a fluid (for example, in a mixture of plasma, anti-coagulant solution, additive solution, and/or saline solution and, which can, for example, include residual platelets and leukocytes).

In a number of representative embodiments, devices, systems and methods studied herein, oxygen is removed from human red blood cell suspension which has been processed for storage and ultimate transfusion. Removal of oxygen from human red blood cell suspension or from a fluid including human red blood cell suspension (for example, blood collected in an anticoagulant solution and processed to remove platelets, white blood cells and other blood constituents) immediately after processing has been shown to extend the shelf life by 30%-100%. Devices or systems for removal of oxygen from a fluid including red blood cells such as red blood cell suspension, red blood cell suspension products or other fluids including red blood cells (sometimes referred to herein collectively as blood) are sometimes referred to herein as oxygen depletion devices or ODDs.

In a number of representative embodiments of ODD devices studied herein, the devices included at least one group or bundle of hydrophobic hollow fiber membranes manifolded within a polymeric (for example, polycarbonate) housing. A biological fluid such as a fluid including red blood cells or a red blood cell suspension flows, for example, through the hollow fibers and oxygen from the blood is transported across the membrane of the hollow fibers to a volume within the housing but outside the fibers, wherein the concentration of oxygen is maintained low. In the studied ODDs, either a carrier or gas sorbent material or flow of an inert carrier or sweep gas (typically, in a countercurrent flow) was used around the annular spaces of the fiber bundle to eliminate gas such as O₂, removed from the fluid within the fibers, thereby maintaining a concentration gradient to drive diffusion.

In alternative embodiments, a biological fluid such as a fluid including red blood cells or a red blood cell suspension can flow through a volume within a housing or enclosure which surrounds a plurality of hollow fibers through which an inert carrier gas can flow, thereby maintaining a concentration gradient to drive oxygen from the fluid including red blood cells flowing external to the fibers into the carrier gas flowing through the fibers.

In still other embodiments, a biological fluid such as a fluid including red blood cells or a red blood cell suspension can flow through a volume within a housing or enclosure in which one or more gas absorbing or gas sorbent members or elements are present. Gas sorbent members such as oxygen sorbent members can, for example, include a sorbent material encased within a gas permeable or gas porous layer or membrane.

In a number of studies, ODDs included polypropylene hollow fiber membranes with an inner diameter of 150 microns and a wall thickness of 25 microns through which a representative fluid including a red blood cell suspension flowed. ODDs were studied with variations in the fiber length, number of fibers, fiber versus sorbent configuration and sorbent versus inert gas configurations for the purpose of studying optimal configuration variables to achieve, for example, more than 95% oxygen removal within a given time constraint as well as to facilitate manufacturability.

Results from studied ODDs were compared to a numerical model of the ODDs. The model was validated by comparison to experimental results. The model was then used to identify parameter values for subsequently designed devices. A number of principles of operation and design are described below.

Oxygen is carried in red blood cell suspension both dissolved in the plasma and attached to the hemoglobin molecules within the red blood cells. Greater than 95% of the oxygen is carried within the red blood cells. To remove the oxygen from the red blood cell suspension, representative ODDs hereof were designed to direct red blood cell suspension flow through the lumens of a bundle of hydrophobic hollow fiber membranes, which were arranged in parallel. The walls of the hollow fiber membrane were very thin and microporous. Because the fiber membrane material can also be hydrophobic, the blood remains in the fiber lumens and the pores remain gas filled. The concentration of oxygen external to the wall of the hollow fibers can be maintained at approximately zero by, for example, sweeping an inert gas (such as nitrogen or argon) within the fiber housing across the outside of the fiber walls, or by positioning within the housing, outside of the fiber walls, an oxygen adsorbing or sorbent material (for example, oxygen-absorbing microporous fibers or particles).

Several devices were fabricated with varying arrangements of oxygen adsorbing particles from Multisorb Technologies of Buffalo, N.Y. US. Such oxygen-absorbing particles have significant carrying capacity for oxygen, and when incorporated in sufficient quantity within the studied ODD, were able to maintain the shell- or outer-side oxygen content of the ODD to less than 0.01%. Oxygen absorbing materials are, for example, described in U.S. Pat. Nos. 6,156,231, 6,248,690, 6,436,872, 6,558,571, 6,899,822, 7,125,498.

The low O₂ concentration outside of the fibers sets up concentration gradients which drive the diffusion of oxygen out of the fluid flowing through the fibers, and across the fiber membrane. The resistance to the oxygen diffusion across the gas filled microporous walls of the fibers is negligible as is the diffusional resistance of either the oxygen from the wall into the gas phase, which is either swept by the inert gas or adsorbed by surrounding sorbent particles. The predominant resistance exists in the boundary layer of the fluid flowing within the hollow fibers. This resistance is governed by the properties of the fluid (viscosity and density), by the flow rate of fluid through the fibers and by the inner diameter of the fibers. The smaller the fiber diameter and the faster the flow rate, the less the resistance to oxygen diffusion through the boundary layer of the fluid adjacent to the fiber wall. However, because of the large quantity of oxygen held within the red blood cell suspension, the amount of time that the red blood cell suspension spends within the fibers (the residence time) affects the total amount of oxygen that can be removed. Longer fibers increase the residence time, but also increase the pressure required to drive red blood cell suspension flow through the device. An increased number of fibers used in the parallel bundle can reduce the overall resistance to red blood cell suspension flow through the device, but increases the size of the housing.

A summary of the physical relations which govern the design and performance of the ODDs including hollow fiber membranes is set forth below. The summary illustrates how pertinent device parameters were modeled and used to effect oxygen removal with several constraint conditions (for example, over a constrained time period, with an acceptable device size and with an acceptable resistance in terms of pressure head required to drive flow).

In that regard, a number of relations were used to model oxygen removal from a standard unit (for example, 400 ml) of banked human red blood cell suspension flowing through a parallel bundle of fibers, assuming the concentration of oxygen outside of the fibers to be zero. The discussion below is divided into four sections which set forth design considerations of the studied representative ODDs.

Processing Time (T_{ODD}).

The processing time T_{ODD} is defined as the time it takes for the ODD to remove the oxygen from a unit of red blood cell suspension.

$${T}_{ODD} = \frac{V}{{Q}_{ODD}} \qquad ( 1 )$$

where V=volume of a unit of red blood cell suspension, and Q_{ODD}=overall device flow rate. For V=400 ml and Q_{ODD}=5 ml/min, for example, T_{ODD}=80 minutes.

Residence Time (λ)

The residence time λ is defined as the time required for a red blood cell suspension to pass down the length of a fiber. The greater the residence time, the more oxygen is removed.

$$λ = \frac{{V}_{fiber}}{{Q}_{fiber}} = π \frac{{R}^{2} L {N}_{f}}{{Q}_{ODD}} \qquad ( 2 )$$

where V_{fiber}=volume within a fiber, Q_{fiber}=flow rate through a single fiber, R=inner radius of a fiber, L=length of a fiber, and N_{f}=number of fibers in the device.

Intuitively, for a given device, equation 2 shows that the device flow rate is inversely proportional to the residence time, meaning that the slower the flow, the more gases are removed. If, for example, a minimum flow rate is set based on a processing time constraint from equation 1, we can use equation 2 to evaluate the effects of the length and number of fibers on processing time. For a set device flow rate, residence time is improved (increased) by increasing the number of fibers, which causes the per fiber flow rate to be decreased. For a set flow rate, decreasing the device length has a negative impact on the residence time. From this equation, it seems counterintuitive that decreasing the fiber inner diameter would have a negative impact by reducing residence time, but if we are looking at the equation from the perspective of a fixed device flow rate, decreasing the radius results in a faster velocity of the red blood cell suspension (flow rate=area x×velocity).

An alternative way of evaluating the effects of the device parameters on the residence time is to define Q_{fiber} based on the equation for Newtonian laminar flow through a tube, which is,

$${Q}_{fiber} = \frac{π ( Δ P ) {R}^{4}}{8 μ L} \qquad ( 3 )$$

where ΔP=pressure drop across fiber length, and μ=viscosity of the red blood cell suspension. Thus residence time can also be written as,

$$λ = \frac{8 μ {L}^{2}}{( Δ P ) {R}^{2}} \qquad ( 4 )$$

From this perspective, it is evident that for a fixed pressure drop across the fibers, decreasing the fiber radius increases residence time by slowing down the fiber flow rate by the power of 4.

Outlet pO₂

The outlet pO₂ can, for example, be estimated by numerically solving a non-linear convective diffusion equation for red blood cell suspension. The non-dimensional form of this equation is,

$$\frac{[ 1 - {{r}^{*}}^{2} ] [ 1 + f ( {p}^{*} ) ] ( \partial {p}^{*} )}{\partial {z}^{*}} = \frac{1}{{r}^{*}} \frac{\partial }{\partial {r}^{*}} ( {r}^{*} \frac{\partial {p}^{*}}{\partial {r}^{*}} ) \qquad ( 5 )$$

Where r*=r/R (a non-dimensional radius), p*=pO₂/pO_{2 in} (a non-dimensional oxygen tension), z*=z [symbol] /VₘₐₓR² (a non-dimensional length), [symbol] is the diffusivity of oxygen in red blood cell suspension, Vₘₐₓ is the velocity of red blood cell suspension at the centerline of the fiber, and f(p*) is a non-dimensional sink function representing the change in oxygen bound to hemoglobin with change in oxygen tension.

Thus, in solving the above equations, parameters which affect the solution for the pO₂ exiting the fibers include:
R (fiber inner radius)
pO_{2 in} (inlet oxygen tension)
[symbol] (diffusivity of oxygen in blood)
Vₘₐₓ (the velocity of blood in the fibers)
f(p*)=(BC_{Hb}/σ)dSO₂/dpO₂ (C_{Hb} is concentration of hemoglobin, B is oxygen capacity of hemoglobin, σ is solubility of oxygen in blood and dSO₂/dpO₂ is the slope of the oxy-hemoglobin dissociation curve)

The equation for Vₘₐₓ can be expressed either in terms of the total flow through the device or the pressure drop across the fibers as,

$${V}_{max} = \frac{{R}^{2}}{4 μ L} Δ P = \frac{2}{π {R}^{2}} \frac{{Q}_{ODD}}{{N}_{f}} \qquad ( 6 )$$

When equation 6 is substituted in to the expression for non-dimensionalizing the independent variable for length, z* becomes,

$${z}^{*} = z \frac{π {N}_{f} D}{2 {Q}_{ODD}} = z \frac{4 μ LD}{{R}^{4} Δ P} \qquad ( 7 )$$

If we want to evaluate the effect of the parameters on the outlet pO₂ based on a specified fixed device flow rate, the middle relation shows that the fiber radius has no effect. The only parameters which affect the result for a specified fixed Q_{ODD} are the diffusivity, the number of fibers, the slope of the oxy-hemoglobin dissociation curve (from equation 5), and the length of the fibers (not from equation 7, but because in the z direction, the solution continues to approach zero oxygen tension as the length is increased).

Q_{ODD} vs. ΔP

The time constraint on the oxygen depletion process ultimately places a minimum limit on the overall device flow rate, which thus governs the parameters which affect the amount of oxygen that can be removed. Once a minimum flow rate is set, the number of fibers and length of fibers can be selected to maximize the removal of oxygen. Although the fiber radius does not have an apparent affect on the amount of oxygen removed for a specified flow rate, it will have an impact on the overall dimensions of the device and the configuration of the process setup in terms of how high the unit of red blood cell suspension will have to be fixed with relation to the ODD to drive red blood cell suspension flow.

The resistance of the ODD to flow can be estimated in terms of the head loss from empirical relationships for viscous energy losses in pipe flow.

$${H}_{L} = f \frac{L {\overline{V}}^{2}}{D 2 g} = \frac{128 μ}{g ρ} \frac{L}{{D}^{4}} \frac{{Q}_{ODD}}{{N}_{f}} \qquad ( 8 )$$

where D=inner diameter of fiber, g=gravitational constant, L=length of fiber, V̅=average velocity of red blood cell suspension flow in a fiber, f=Darcy friction factor=64/Re, Re=Reynolds number=ρ V̅D/μ, and ρ=fluid density.

For 8000 fibers with a length of 30 cm at a red blood cell suspension flow rate of 5 g/min and a fiber ID of 150 microns, the head loss would be approximately 20 cm. For a fiber ID of 240 microns, the head loss would only be 8 cm. This difference in height is not great enough to warrant the requirement of a fiber ID of 150 microns versus 240 microns. However, the smaller ID does allow for a tighter packing density, and a smaller volume of red blood cell suspension that must be drained from the device at the end of the process.

In several studies of the devices, systems and methods hereof, a series of 9 ODD devices were fabricated. Table 1 below sets forth a summary of the specifications of the studied devices.

TABLE 1

| | External Gas | External Gas |
|---|---|---|
| Prototype Specifications | Pathways | Pathways |
| Prototype Serial #: | BAL00001 | BAL00002 |
| Fiber Type: | Celgard | Celgard |
| | 200/150-66FPI | 200/150-66FPI |
| Number of Fibers: | 5000 | 5000 |
| Active Length of Fibers | 13 | 28 |
| (cm): | | |
| Fiber OD (microns): | 200 | 200 |
| Fiber ID (microns): | 150 | 150 |
| Total Length of Fibers | 15 | 30 |
| Active Fiber Surface | 0.4084 | 0.8796 |
| Area (m2): | | |

| | Center Core | 10 Individual Bundles |
|---|---|---|
| Prototype Specifications | 125 grams Sorbent | 200 grams Sorbent |
| Prototype Serial #: | BAL00003 | BAL00004 |
| Fiber Type: | Celgard | Celgard |
| | 200/150-66FPI | 200/150-66FPI |
| Number of Fibers: | 5000 | 5000 |
| Active Length of Fibers | 28 | 28 |
| (cm): | | |
| Fiber OD (microns): | 200 | 200 |
| Fiber ID (microns): | 150 | 150 |
| Total Length of Fibers | 30 | 30 |
| Active Fiber Surface | 0.8796 | 0.8796 |
| Area (m2): | | |

| Prototype Specifications | Center Core—2 O₂ Sachets |
|---|---|
| Prototype Serial #: | BAL00005 |
| Fiber Type: | Celgard |
| | 200/150-66FPI |
| Number of Fibers: | 8000 |
| Active Length of Fibers | 28 |
| (cm): | |
| Fiber OD (microns): | 200 |
| Fiber ID (microns): | 150 |
| Total Length of Fibers | 30 |
| Active Fiber Surface | 1.407 |
| Area (m2): | |

| | 1″Center | 1″Center |
|---|---|---|
| | Core—118 grams | Core—118 grams |
| | Pre-activated | Pre-activated |
| Prototype Specifications | Sorbent | Sorbent |
| Prototype Serial #: | BAL00009 | BAL00010 |
| Fiber Type: | Celgard | Celgard |
| | 200/150-66FPI | 200/150-66FPI |
| Number of Fibers: | 8000 | 8000 |
| Active Length of Fibers | 28 | 28 |
| (cm): | | |
| Fiber OD (microns): | 200 | 200 |
| Fiber ID (microns): | 150 | 150 |
| Total Length of Fibers | 30 | 30 |
| Active Fiber Surface | 1.407 | 1.407 |
| Area (m2): | | |
| Prototype Serial #: | BAL00011 | BAL00012 |
| Fiber Type: | Celgard | Celgard |
| | 200/150-66FPI | 200/150-66FPI |
| Number of Fibers: | 8000 | 8000 |
| Active Length of Fibers | 28 | 28 |
| (cm): | | |
| Fiber OD (microns): | 200 | 200 |
| Fiber ID (microns): | 150 | 150 |
| Total Length of Fibers | 30 | 30 |
| Active Fiber Surface | 1.407 | 1.407 |
| Area (m2): | | |

All studied devices were fabricated with Celgard polypropylene microporous hollow fiber membrane fabrics (available from Celgard, LLC of Charlotte, N.C.) of 66 fibers per inch, with a fiber outer diameter of 200 microns and an inner diameter of 150 microns (Celgard 200/150-66FPI).

A number of variables or specifications, including number of fibers, fiber and overall device dimensions, shell side arrangement (that is, inert gas flow versus adsorbent particles), and arrangement of adsorbent particles relative to the fibers were varied between the studied devices.

In several representative embodiments, the fabrication of the studied ODDs began with constructing an annular fiber bundle by, for example, concentrically wrapping hollow fiber membrane fabric around a removable center core. A removable center core can, for example, provide support to the fiber for potting and also, for example, provide an area for placement of an oxygen sorbent material (Multisorb Technologies, Inc. Buffalo N.Y.) within the device. A two-piece reusable mold made from Delrin® (an acetal resin, available from E.I. DuPont De Nemours and Company of Wilmington, Del.) was used to control position of the fibers in the ODD housing. The fibers were potted and molded at both ends of the ODD device by injecting a two-part polyurethane adhesive (available from Vertellus of Greensboro, N.C.) into the mold. The mold was removed after the adhesive was allowed to dry, and the potted fibers were then exposed and tomed in a fixture to establish a common pathway between all fibers. The ODD further included a main housing and two end caps which were manufactured from a polymeric material such as polycarbonate (available from Professional Plastics, Inc. of Albany, N.Y.).

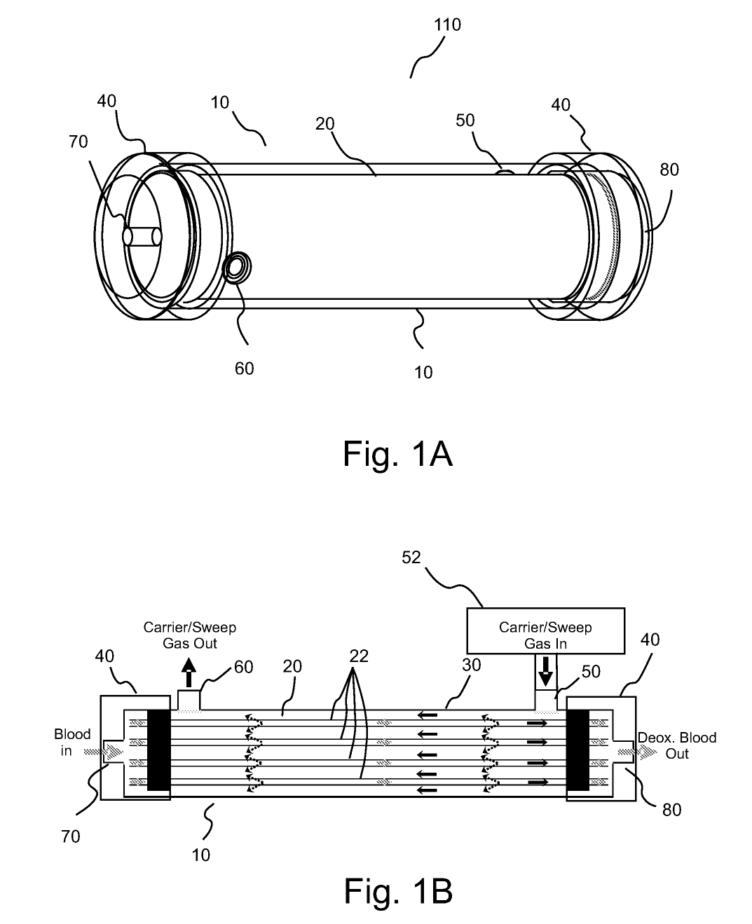

Device 10 (see FIGS. 1A and 1B), which corresponds, for example, to labeled devices BAL0001 and BAL0002 in Table 1, included a hollow fiber bundle 20 comprising a plurality of hollow fibers 22 (see FIG. 1B) as described above within a housing 30. As also described above, housing 30 include an end cap 40 on each end thereof. Housing further included an inlet 50 through which an inert carrier gas (argon gas in the studies) could enter the housing and an outlet 60 through which the inert carrier gas could exit the housing after flowing around hollow fibers 22 to remove gas such as O₂ diffusing from the red blood cell suspension through the microporous walls of hollow fibers 22. A relatively small pressurized vessel 52 (illustrated schematically in FIG. 1B) of carrier gas can, for example, be provided with device 10. Red blood cell suspension (or a red blood cell suspension product fluid) entered hollow fiber bundle 20 via an inlet 70 through which the red blood cell suspension was distributed to hollow fibers 22 of hollow fiber bundle 20. Deoxygenated red blood cell suspension exited hollow fibers 22 of hollow fiber bundle 20 via a common outlet 80. Oxygen diffusing through the microporous walls of hollow fibers 22 is represented schematically by dashed arrows in FIG. 1B. BAL0001 was manufactured with an active fiber length of 13 cm, while BAL0002 was manufactured with an active fiber length of 28 cm. Neither BAL0001 nor BAL0002 included an oxygen sorbent.

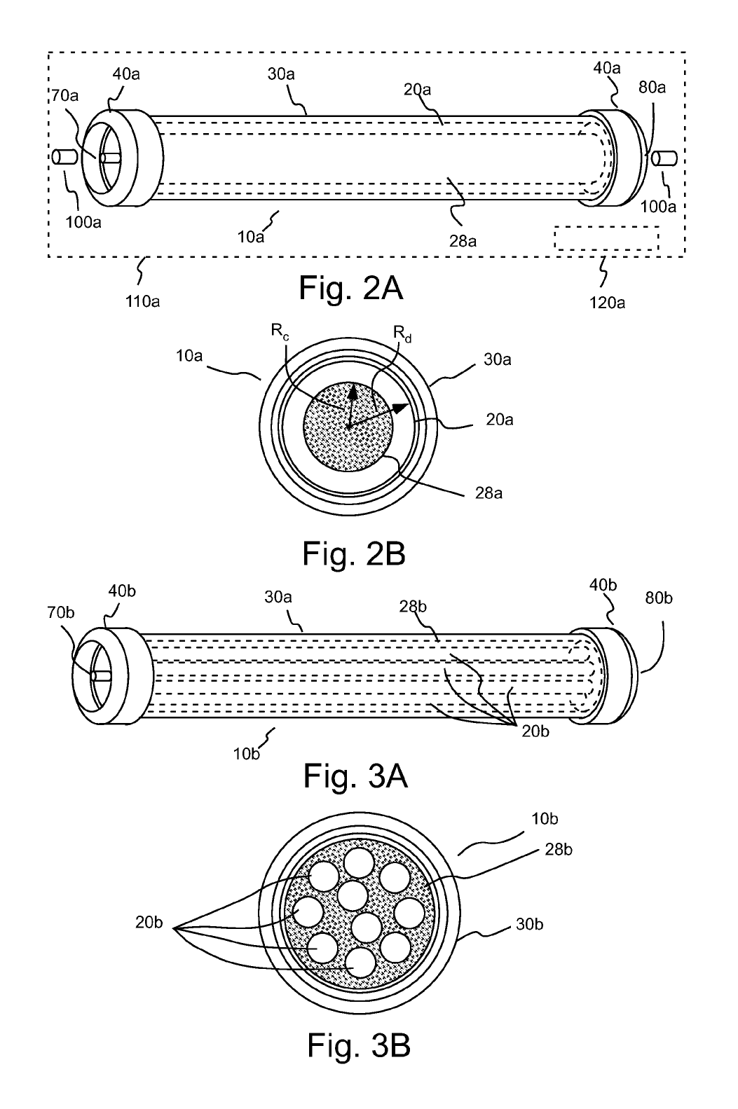

FIGS. 2A and 2B illustrate an embodiment of a device 10a that is representative of the device labeled BAL0003 in Table 1. Device 10 included a hollow fiber bundle 20a including a plurality of hollow fibers (not depicted individually in FIGS. 2A and 2B) within a housing 30a. Device 10 further included a generally centrally positioned (relative to fiber bundle 20a) oxygen sorbent material(s) 28a. In the case of BAL0003, a center core of hollow fiber bundle 20a was filled with 125 grams of sorbent material 28a. Similar to device 10, housing 30a of device 10a included an end cap 40a on each end thereof. Red blood cell suspension entered hollow fiber bundle 20a via an inlet 70a through which the red blood cell suspension was distributed to the individual hollow fibers of hollow fiber bundle 20a, while deoxygenated red blood cell suspension exited hollow fiber bundle 20a via a common outlet 80a.

FIGS. 3A and 3B illustrate an embodiment of a device 10b that is representative of the device labeled BAL0004 in Table 1. Device 10b included a plurality (ten bundles in the studied embodiments) of hollow fiber bundles 20b (each including a plurality of hollow fibers (with 500 fibers each in the studied embodiments), which are not shown individually in FIGS. 3A and 3B) within a housing 30b. A total of 200 grams of a sorbent material 28b was placed in the volume between individual hollow fiber bundles 20b. Similar to devices 10a and 10b, housing 30b of device 10b included an end cap 40b on each end thereof. Red blood cell suspension entered hollow fiber bundle 20b via an inlet 70b through which the red blood cell suspension was distributed to the individual hollow fibers of hollow fiber bundles 20b, while deoxygenated red blood cell suspension exited hollow fiber bundles 20b via a common outlet 80b.

BAL0005 was constructed with a center core (as discussed in connection with FIGS. 2A and 2B) packed with varying amounts (see Table 1) of O₂ un-activated sorbent sachet material (Multisorb).

BAL0009, BAL0010, BAL0011 and BAL0012 were each constructed with a center core (as discussed in connection with FIGS. 2A and 2B) packed with 118 grams of pre-activated sorbent sachets (Multisorb DSR#5353C).

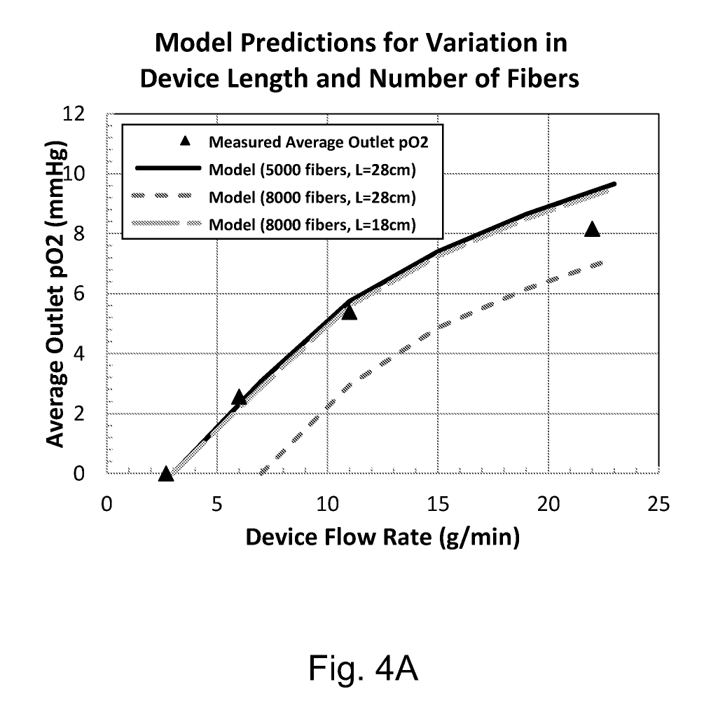

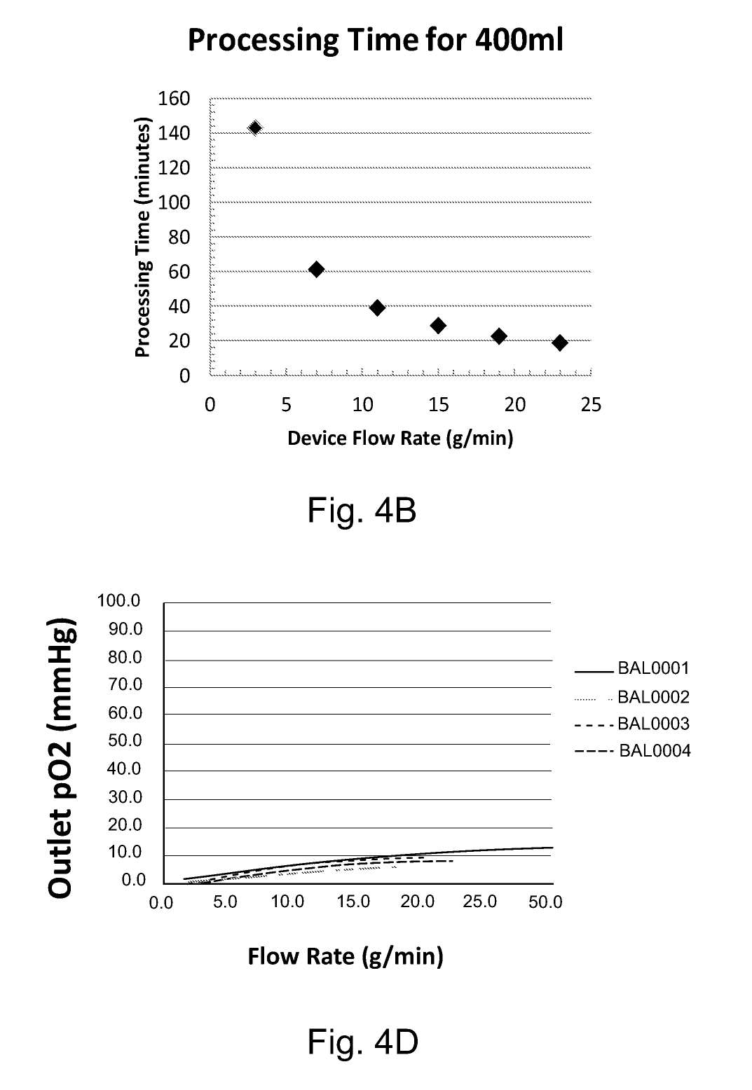

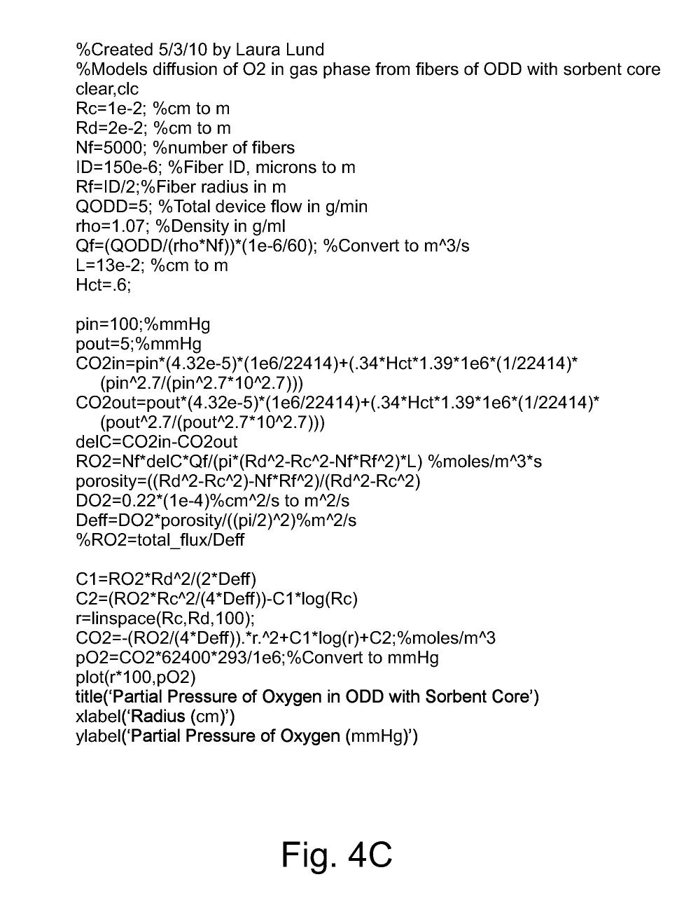

In the design of the devices of Table 1, the predicted results from the numerical model described above were compared with experimental results for BAL0004 as illustrated in FIGS. 4A and 4B. The model was then used to select a larger number of fibers for other devices.

Estimates of the shell side resistance to oxygen flux (that is, the diffusion of oxygen from the fiber walls to the sorbent filled core of the device) indicate that it is negligible in comparison with the resistance to oxygen flux in the fluid boundary layer within the fibers. A consequence of the shell side resistance being negligible is that the arrangement of sorbent relative to the fibers can be chosen based on simplification of manufacturability, and that the chosen configuration will not have a significant negative impact on the amount of oxygen removal relative to another configuration. To confirm such estimates, a model of the shell side oxygen tension as a function of radial distance from a sorbent core was developed and compared to preliminary estimates as well as to bench test results of different sorbent configurations.

Preliminary Estimation of Shell Side Resistance.

As, for example, red blood cell suspension is flowing through the device, the radial diffusion of oxygen from the red blood cell suspension filled fibers will be equal to the amount of oxygen diffusing from the fiber walls to the sorbent core. The amount of oxygen diffusion, J, is directly proportional to the oxygen concentration gradient in the direction of diffusion, thus

$$J = {( D \frac{d C}{d r} )}_{blood} = {( D \frac{d C}{d r} )}_{gas} \qquad ( 9 )$$

where D is the constant of proportionality which represents the diffusivity of oxygen in either red blood cell suspension or the gas surrounding the fibers (which would essentially be nitrogen). Rearranging the above components gives

$$\frac{d {C}_{blood}}{d {C}_{gas}} = ( \frac{{D}_{gas}}{{D}_{blood}} ) ( \frac{d {r}_{blood}}{d {r}_{gas}} ) \qquad ( 10 )$$

Equation 10 can be used to give an estimate of the radial concentration gradient of oxygen in the red blood cell suspension flowing through the fibers relative to average radial oxygen concentration gradient in the gas filled shell surrounding the fibers. Using the diffusivity of oxygen in nitrogen under atmospheric conditions, which is 0.22 cm²/s, and the diffusivity of oxygen in red blood cell suspension at body temperature, which is approximately 1×10⁻⁵ cm²/s, and approximating dr_{blood} to be 75 microns and dr_{gas} to be 1 cm, the ratio of dC_{blood} to dC_{gas} is 150. In other words, the concentration gradient of oxygen from the center of the fiber to the fiber wall is 150 times greater than the concentration gradient from the fiber wall to the sorbent core. For steady state oxygen flux in both phases, this shows that the resistance in the gas phase is roughly 150 times smaller than in the red blood cell suspension flow and can thus be considered to be negligible.

Model of Shell Side Oxygen Concentration Profile.

To validate the above estimation, a model of the shell side oxygen flux was developed using a mass balance equation applied to the shell side of the device. Assuming the shell side of the device to be sealed with a sorbent filled core, and the rate sorbent reaction with oxygen to be much faster than the diffusion process (which has previously been demonstrated for the sorbents studied), the mass balance equation for oxygen in the gas phase surrounding the fibers and filling the shell is reduced to,

$${D}_{eff} \frac{1}{r} \frac{\partial }{\partial r} ( r \frac{\partial C}{\partial r} ) + {R}_{{O}_{2}} = 0 \qquad ( 11 )$$

where C is the concentration of oxygen in moles/liter, R_{O2} represents the local rate of oxygen “production” by the fibers (that is, the amount of oxygen diffusing from the fiber walls), and D_{eff} is the effective diffusivity of oxygen. The effective diffusivity takes into account the tortuosity of the path for diffusion of an oxygen molecule from the fiber wall around the fibers to the core, as well as the porosity of the fiber bundle through which the oxygen must diffuse, and is determined from the equation,

$${D}_{eff} = \frac{ɛ}{{a}^{2}} {D}_{{O}_{2} - {N}_{2}} \qquad ( 12 )$$

where ε is the porosity of the fiber bundle, ‘a’ is the tortuosity (the length of the path a molecule of oxygen must travel around a fiber relative to the direct path, which is just half the circumference divided by the fiber outer diameter, or π/2), and D_{O2-N2} is the diffusivity of oxygen in nitrogen.

The solution of the mass balance equation requires two boundary conditions. At r=R_{d} (the outer shell radius), the diffusion of oxygen must be zero, or dC(r=R_{d})/dr=0, and at the core surface, the concentration of oxygen is assumed to be zero, or C(r=R_{c})=0. As shown in the FIG. 2A, R_{c} is the radius of the sorbent core, and R_{d} is the radius of the shell of the device (or the outer radius of the hollow fiber membranes).

Using these boundary conditions, the solution of the mass balance equation is,

$$\begin{matrix}C ( r ) = ( \frac{- {R}_{{O}_{2}}}{4 {D}_{eff}} ) {r}^{2} + {C}_{1} ln ( r ) + {C}_{1} & ( 13 ) \\ {C}_{1} = \frac{{R}_{{O}_{2}} {R}_{d}^{2}}{2 {D}_{eff}} & ( 14 ) \\ {C}_{1} = \frac{{R}_{{O}_{2}} {R}_{c}^{2}}{4 {D}_{eff}} - {C}_{1} ln ( {R}_{c} ) & ( 15 )\end{matrix}$$

The rate of oxygen transferred to the gas phase per unit volume, R_{O2}, was calculated based on the total amount of oxygen removed from the red blood cell suspension per unit time divided by the volume of air space in to which the oxygen was transferred, which is

$${R}_{{O}_{2}} = \frac{( {C}_{{b}_{in}} - {C}_{{b}_{out}} ) {Q}_{T}}{π ( {R}_{d}^{2} - {R}_{o}^{2} - {N}_{f} {R}_{f}^{1} ) L} \qquad ( 16 )$$

where Cbᵢₙ is the concentration of oxygen in the red blood cell suspension entering the fibers and Cbₒᵤₜ is the concentration desired at the outlet of the device, Q_{T} is the total red blood cell suspension flow rate through the device, N_{f} is the number of fibers, and R_{f} is the outer radius of a fiber.

This solution was programmed using MATLAB® computer software available from The Mathworks Inc of Natick, Mass. The code used is shown in FIG. 4C and provides the parameter values, units, and conversions used. The output of the model showed an oxygen profile with a value of zero at r=R_{d}, and 0.2 mmHg at r=R_{c}. If the partial pressure of oxygen in the red blood cell suspension at the inlet of the fibers is approximately 100 mmHg, the radial distribution of oxygen tension within the fibers would be a maximum of approximately 100 mmHg, decreasing along the length of the fibers. The ratio of an average of this distribution relative to the 0.2 mmHg modeled distribution on the shell side is consistent with that of preliminary estimates, again indicating a negligible resistance on the shell side to oxygen flux, and, therefore, that the amount of oxygen removed by the ODD is independent of the configuration of the sorbent relative to the fibers.

This result assumes that the amount of sorbent contained in the core is sufficient to react with all of the oxygen to be removed and that the reaction rate remains effectively instantaneous over the entire removal time period. The specifications for the sorbents used in the studied devices indicated that the oxygen capacity of the amount of sorbent used in the studies was several times greater what was necessary and that the reaction rates should remain high.

Prototype Test Results.

The results of tests of devices BAL0002 through BAL0004 allow a comparison of oxygen removal as a function of the total red blood cell suspension flow rate through the device for devices with the shell side open to an argon sweep gas (BAL0002) versus a device with a sealed shell side and sorbent core (BAL0003), and with a sealed shell and sorbent “rods” evenly distributed among the fiber bundle (BAL0004). A cross-section of BAL0003 and BAL0004 is set forth in FIGS. 2B and 3B, respectively, which diagram the sorbent arrangements. The test results are plotted in FIG. 4D, and include the results of BAL0001 which was fabricated with a shorter fiber length than devices BAL0002, BAL0003, and BAL0004. The results show that at a flow rate of approximately 5 g/min, all the devices removed oxygen to targeted levels. The device with the argon sweep or carrier gas performed slightly better than the devices with the sorbent. However the differences were not statistically significant. The results indicate that the shell side arrangement for the studied devices has little to no effect on the oxygen removal capacity of the device (for the quantity of sorbents used), as anticipated from the mathematical model.

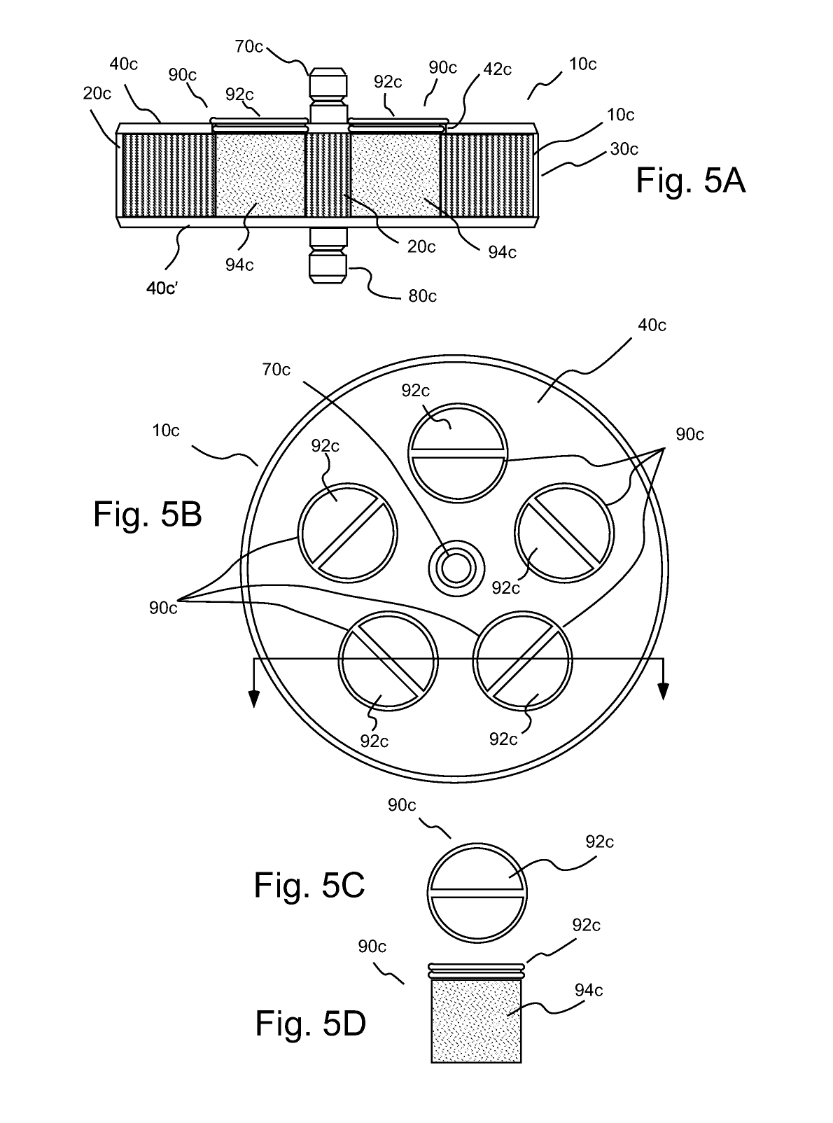

As clear to one skilled in the art, many configurations of hollow fibers and/or gas sorbent materials are possible. For example, FIGS. 5A through 5D illustrate an embodiment of a device 10c including a plurality of hollow fiber bundles 20c within a housing 30c. As described above, housing 30c includes end caps 40c and 40c′ on each end thereof. An inlet 70c is in fluid connection with hollow fiber bundles 20c (comprising relatively short length fibers 22c compared to the diameter of housing 30c) at a first end of housing 30c, through which a biological fluid such as a fluid including red blood cells enters hollow fiber bundles 20c, and an outlet 80c at a second end of housing, through which deoxygenated blood exits hollow fiber bundles 20c. A plurality of sorbent cartridges 90c including an upper or cap member 92c and a sorbent volume 94c are connectible in a modular fashion within housing 30c via openings 42c in end cap 40c.

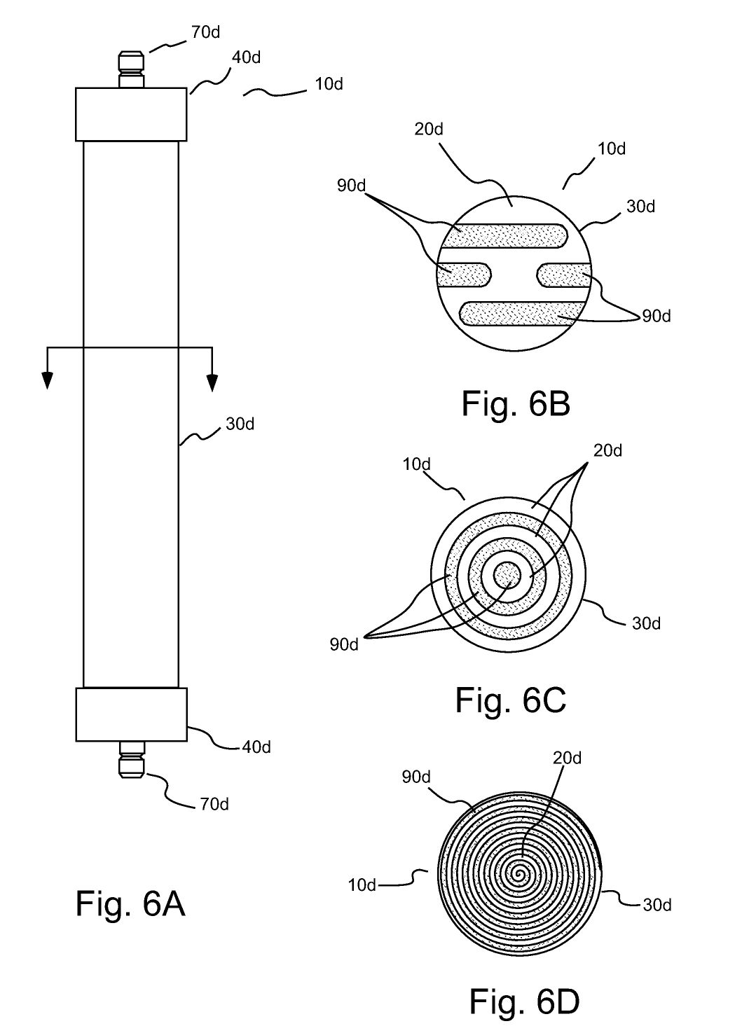

FIGS. 6A through 6D illustrate a device 10d including one or more hollow fiber bundles 20d (see FIGS. 6B through 6D) within a housing 30d. As described above, housing 30d includes end caps 40d on each end thereof. An inlet 70d is in fluid connection with hollow fiber bundle(s) 20d (comprising relatively long length fibers (not shown individually) compared to the diameter of housing 30d) at a first end of housing 30d, through which a biological fluid such as a fluid including red blood cells enters hollow fiber bundle(s) 20d, and an outlet 80d at a second end of housing, through which deoxygenated fluid exits hollow fiber bundle(s) 20d.

In the embodiment of FIG. 6B, a plurality of gas sorbent material volumes 90d, which are elongated in a direction perpendicular to the orientation of the hollow fibers, are positioned within voids within or between hollow fiber bundle(s) 20d. In the embodiment of FIG. 6C a plurality of hollow fiber bundles 20d and sorbent volumes 90d are arranged concentrically. In the embodiment of FIG. 6D, a generally spiraled hollow fiber bundle or fiber membrane fabric 20d is adjacent a similarly spiraled volume of sorbent material 90d.

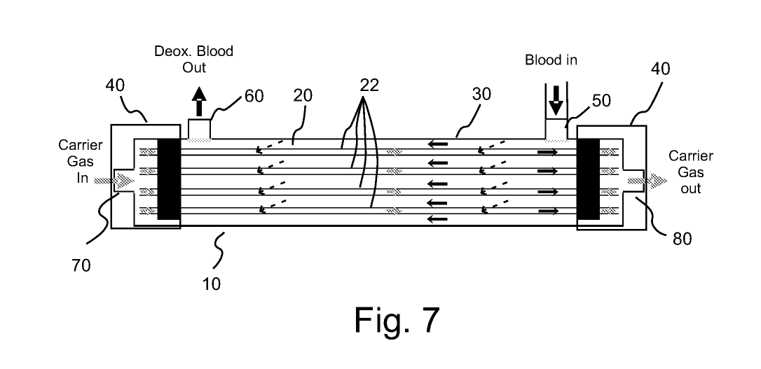

In the representative embodiments described above, red blood cell suspension flows through the lumens of a plurality of hollow fibers. As illustrated schematically in FIG. 7 for the representative embodiment of device 10, red blood cell suspension or other biological fluid can flow alternatively through the volume within housing 10 (or other housing or enclosure) which surrounds hollow fibers 22 of hollow fiber bundle 20. In the embodiment of FIG. 7, an inert carrier gas enters inlet 70 to flow through hollow fibers 22 and exits via outlet 80. A concentration gradient is created by the flow of carrier gas through hollow fibers 22 to drive oxygen from the fluid flowing external to hollow fibers 22 into the carrier gas flowing through hollow fibers 22.

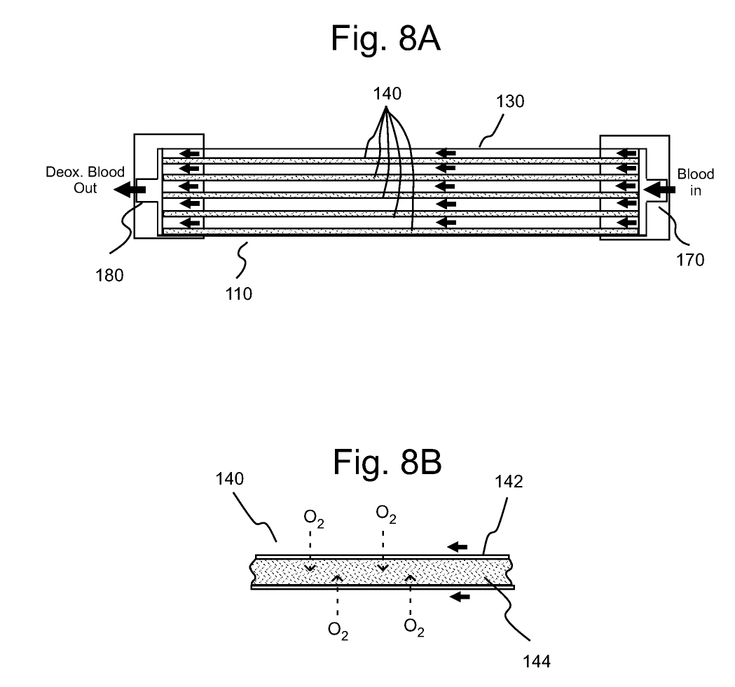

FIG. 8A illustrates another embodiment of an oxygen depletion device 110 which includes a plurality of generally cylindrical gas sorbent elements 140 extending through a housing 130, A biological fluid such as a fluid including red blood cells enters housing 130 via inlet 170, flows through the volume exterior to sorbent elements 140, and deoxygenated fluid exits housing 130 via outlet 180. Oxygen from the fluid is absorbed by sorbent elements 140. As illustrated in FIG. 8B, sorbent elements 140 can, for example, include a gas permeable or microporous layer 142 (for example, as described above for hollow fiber membranes) encompassing a sorbent material 144 (for example, a particulate of fibrous sorbent material). Gas from the fluid, specifically O₂ diffuses through layer 142 into sorbent material 144, which is illustrated by dashed arrows in FIG. 8B.

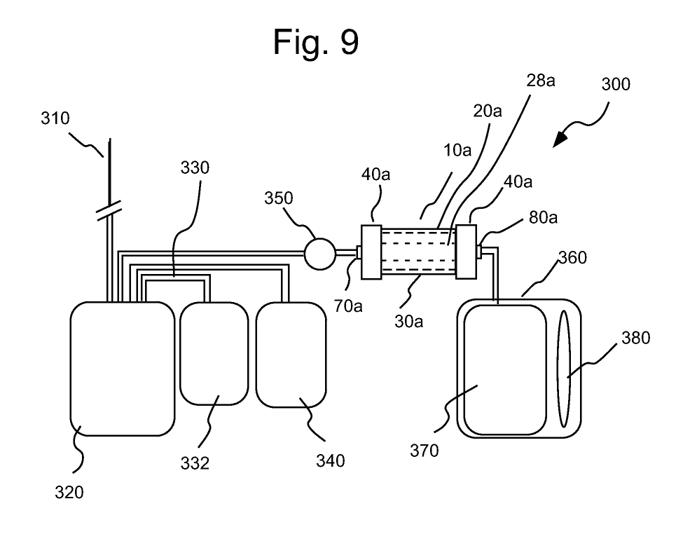

In several embodiments, oxygen depletion devices hereof (which can, for example, be disposable) can be readily incorporated into existing blood bank processing and/or storage systems to, for example, deplete red cells of oxygen (and/or other gases) prior to storage within a storage container. FIG. 9 illustrates a representative embodiment of a system 300 which is, for example, in fluid connection with a phlebotomy needle 310 for drawing blood from a patient (for example, 400 ml). The blood can, for example, pass to an initial collection container or bag 320 that can, for example, include an anticoagulant and/or other additives. The blood can be processed via a system 330 such that at least part of the plasma is removed therefrom, which can be stored in a plasma container or bag 332. Removed plasma can, for example, be at least partially replaced by lower viscosity preservative solution (for example, 200 ml in a representative example) such as an oxygen free additive solution from a container 140. See, for example, Published U.S. Patent Application Nos. 2003/0153074 and 2005/0208462. An oxygen depletion device such as device 10a can, for example, be incorporated into system 300 downstream (for example, below) a leukoreduction filter or LRF 350, thereby imposing a serial resistance to that of LRF 350. The flow through device 10a or other oxygen depletion device can, for example, be gravity driven or can be pumped. Device 10a or other oxygen depletion device can, for example, reduce the hemoglobin saturation of red blood cells to a predetermined level (for example, below 2%) just prior to the red cells flowing into a an oxygen impermeable blood storage bag 360. In the illustrated embodiment, the processed fluid including red blood cells is contained, for example, within a PVC bag 370 within oxygen impermeable blood storage bag 360. An oxygen sorbent material 380 (for example, as described above) can also be placed within oxygen impermeable blood storage bag 360. Oxygen impermeable blood storage bag 360 can also be flushed with an inert gas such as argon prior to storage of the processed fluid/blood therein to remove oxygen therefrom. Either alone or in conjunction with using a preservative solution, the desaturation of the red cells prior to storage can significantly extend the shelf life of stored blood. Moreover, the incorporation of the oxygen depletion devices hereof into system 300 and/or other blood processing systems adds little time (for example, less than 10%) to the current processing time for blood storage.

The oxygen depletion devices hereof can, for example, be readily incorporated as a disposable component of existing blood bank processing systems designed to remove oxygen from red blood cell suspension prior to storage. Well known connector systems 100a (see FIG. 2A) such as luer connectors (which can, for example, be attachable to or formed upon the oxygen depletion devices hereof) can be used to connect the oxygen depletion devices to tubing of such systems. Oxygen depletion devices such as device 10A can, for example, be provided in a sealed container 110a (illustrated schematically in dashed lines in FIG. 2A) wherein at least the fluid contacting portions are in a sterile state. One or more other processing or other components 120a (illustrated schematically in dashed lines in FIG. 2A) such as tubing, connectors, etc. can be provided as a kit with device 10a or other oxygen depletion device hereof.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A system for reducing the concentration of oxygen in a fluid comprising red blood cells, comprising:
a housing,
a plurality of hollow tubes extending within the housing and adapted for flow of the fluid therethrough, each tube comprising an inlet and an outlet, and
a carrier system that reduces the concentration of oxygen at an exterior surface of the tubes to facilitate transport of oxygen from the fluid flowing through the tubes to an exterior of the tubes.

2. The system of claim 1 wherein the carrier system comprises a fluid inlet in fluid connection with the housing, a fluid outlet in fluid connection with the housing and a system to circulate a fluid through a volume of the housing exterior to the tubes.

3. The system of claim 2 wherein the fluid that circulates through the volume of the housing comprises a gas other than oxygen.

4. The system of claim 1, wherein the plurality of hollow tubes are microporous tubes having tube diameters ranging from 150 microns to 200 microns.

5. The system of claim 1 wherein the plurality of hollow tubes are microporous tubes having pore diameters in the range of approximately 0.01 to 0.5 microns.

6. The system of claim 1 wherein the plurality of hollow tubes are microporous tubes having pore diameters in the range of approximately 0.1 to 0.4 microns.

7. The system of claim 1, wherein the plurality of tubes range in number from 5000 to 8000 tubes.

8. The system of claim 1, wherein the plurality of tubes range in length from 10 cm to 50 cm.

9. The system of claim 3, wherein the fluid inlet in fluid connection with the housing is in fluid communication with a pump or in fluid communication with a source of the gas other than oxygen that is pressurized, or the fluid outlet in fluid connection with the housing is in fluid communication with a vacuum source to circulate the gas other than oxygen through the volume of the housing.

10. The system of claim 1 wherein the carrier system comprises an oxygen absorbing material.

11. The system of claim 10 wherein the oxygen absorbing material is immobilized on an exterior surface of at least a portion of the tubes.

12. The system of claim 10 wherein the oxygen absorbing material is positioned within a volume of the housing exterior to the tubes.

13. The system of claim 10 wherein a number of the tubes are arranged in a group and the absorbing material surrounds at least a portion of a length of the group.

14. The system of claim 10 wherein a number of the tubes are arranged in a plurality of groups and the absorbing material surrounds at least a portion of a length of each of the plurality of groups.

15. The system of claim 10 wherein a number of the tubes are arranged around a perimeter of the absorbing material over at least a portion of a length of the tubes.

16. The system of claim 1 wherein the system is connectible within a fluid path of a fluid processing system.

17. The system of claim 1 wherein the fluid comprises a red blood cell suspension.

18. A method for reducing the concentration of oxygen in a fluid comprising red blood cells, comprising:
flowing the fluid through a plurality of hollow tubes extending within a housing, a carrier system being in operative connection with the tubes to reduce the concentration of oxygen at an exterior surface of the tubes to facilitate transport of oxygen from the fluid flowing through the tubes to an exterior of the tubes.

19. The method of claim 19 wherein the fluid comprises a red blood cell suspension.

20. A system for reducing the concentration of oxygen in a fluid comprising red blood cells, comprising:
a housing,
a plurality of hollow tubes extending within the housing and comprising an oxygen absorbent material within the tubes,
an inlet in fluid connection with a volume of housing exterior to the tubes for entry of the fluid into the housing, and
an outlet in fluid connection with a volume of housing exterior to the tubes for exit of the fluid from the housing.
